(19) United States
(12) Patent Application Publication
(10) Pub. No.: US 2024/0318155 A1
(43) Pub. Date: Sep. 26, 2024

(54) NUCLEIC ACID-GUIDED NICKASES

(71) Applicant: Inscripta, Inc., Pleasanton, CA (US)

(72) Inventors: Juhan Kim, Boulder, CO (US); Benjamin Mijts, Boulder, CO (US)

(73) Assignee: Inscripta, Inc., Pleasanton, CA (US)

(21) Appl. No.: 18/613,420

(22) Filed: Mar. 22, 2024

Related U.S. Application Data
(63) Continuation of application No. 17/676,218, filed on Feb. 20, 2022, now Pat. No. 11,965,186.
Application No. 17/676,218 is a continuation of application No. 17/463,581, filed on Sep. 1, 2021, now Pat. No. 11,268,078.
(60) Provisional application No. 63/133,502, filed on Jan. 4, 2021.

(51) Int. Cl. C12N 9/22; C12N 15/11; C12N 15/90

(52) U.S. Cl. CPC C12N 9/22; C12N 15/11; C12N 15/907; C12Y 301/21004; C12N 2310/20; C12N 2800/80

(57) ABSTRACT

The present disclosure provides engineered nucleic acid-guided nickases and optimized scaffolds for making rational, direct edits to nucleic acids in live cells.

### RELATED CASES

This application is a continuation of U.S. Ser. No. 17/463.581, filed 1 Sep. 2021, now allowed; which claims priority to U.S. Ser. No. 63/133,502, filed 4 Jan. 2021, which are incorporated herein in their entirety.

### FIELD OF THE INVENTION

The present disclosure provides engineered nucleic acid-guided nickases and optimized scaffolds for making rational, direct edits to nucleic acids in live cells.

### INCORPORATION BY REFERENCE

Submitted with the present application is an electronically filed sequence listing via EFS-Web as an ASCII formatted sequence listing, entitled “INSC094US_seq_list_20210818”, created Aug. 18, 2021, and 77,000 bytes in size. The sequence listing is part of the specification filed herewith and is incorporated by reference in its entirety.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow manipulation of gene sequence; hence, gene function. These nucleases include nucleic acid-guided nucleases. The range of target sequences that nucleic acid-guided nucleases can recognize, however, is constrained by the need for a specific PAM to be located near the desired target sequence. Providing nucleases with altered PAM preferences and/or altered activity or fidelity may one goal of nuclease engineering. Another goal of engineering nucleic acid-guided nucleases may be to create nickases, which create single-strand breaks rather than double-strande breaks. Such changes may increase the versatility of nucleic acid-guided nucleases for certain editing tasks.

There is thus a need in the art of nucleic acid-guided nuclease gene editing for novel nucleases or nickases with varied PAM preferences, varied activity in cells from different organisms, different cutting motifs and/or altered enzyme fidelity. The novel MAD nickases described herein satisfy this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Thus, the present disclosure embodies a nucleic acid-guided nickase selected from the following nickases: MAD2019-H848A, having the amino acid sequence of SEQ ID NO: 3; and MAD2019-N871A, having the amino acid sequence of SEQ ID NO: 4.

In some aspects, the MAD2019-H848A and MAD2019-N871A nickases are in a nucleic acid-guided nickase editing system with a gRNA scaffold having a nucleic acid sequence of SEQ ID NO: 9, SEQ ID NO: 10, SEQ ID NO: 11, SEQ ID NO: 17, SEQ ID NO: 18. SEQ ID NO: 19, SEQ ID NO: 20, SEQ ID NO: 21, SEQ ID NO: 22, SEQ ID NO: 23, or SEQ ID NO: 24; and in some aspects, the MAD2019-H848A and MAD2019-N871A nickases are in a nucleic acid-guided nickase editing system with a native CRISPR repeat having a nucleic acid sequence of SEQ ID NO: 7 and a native tracr RNA having a nucleic acid sequence of SEQ ID NO:8.

In yet other aspects, the MAD2019-H848A and MAD2019-N871A nickases are in a nucleic acid-guided nickase editing system comprising a guide RNA wherein the guide comprises from 5′ to 3′ a guide sequence, a homology region and SEQ ID NO: 30.

In addition, the present disclosure embodies a nucleic acid-guided nickase selected from the following nickases: MAD2017-H847A, having the amino acid sequence of SEQ ID NO: 5; and MAD2017-N870A, having the amino acid sequence of SEQ ID NO: 6.

In some aspects of this embodiment, the MAD2017-H847A and MAD2017-N870A nickases are in a nucleic acid-guided nickase editing system with a gRNA scaffold having a nucleic acid sequence of SEQ ID NO: 14. SEQ ID NO: 15, SEQ ID NO: 16, SEQ ID NO: 25, SEQ ID NO: 26, or SEQ ID NO: 27; and in some aspects, the MAD2017-H871A and MAD2017-N870A nickases are in a nucleic acid-guided nickase editing system with a native CRISPR repeat having a nucleic acid sequence of SEQ ID NO: 12 and a native tracr RNA having a nucleic acid sequence of SEQ ID NO:13.

In yet other aspects, the MAD2017-H847A and MAD2017-N870A nickases are in a nucleic acid-guided nickase editing system comprising a guide RNA wherein the guide comprises from 5′ to 3′ a guide sequence, a homology region and SEQ ID NO. 30.

These aspects and other features and advantages of the invention are described below in more detail.

### BRIEF DESCRIPTION OF THE FIGURES

FIGS. 1A and 1B are exemplary workflows for screening for optimized scaffolds to be used with nucleic acid-guided nickases.

FIGS. 2A, 2B and 2C are heat maps showing the digestion patterns resulting from using the MAD2019 nuclease and three different guide RNAs with a plasmid target with degenerate PAM sequences at both 37° C. and 45° C.

FIGS. 3A, 3B and 3C are heat maps showing the digestion pattern resulting from using the MAD2017 nuclease and three different guide RNAs with a plasmid target with degenerate PAM sequences at both 37° C. and 45° C.

FIGS. 4A and 4B are heat maps showing the results where two sgRNA scaffolds for each of the MAD2019 and MAD107 nucleases were used to test double-strand break formation on a synthetic GFP locus integrated into HEK293T cells.

FIG. 5A shows the editing performance of the CF MAD2019 nickase and various scaffolds with two CREATE Fusion (CF) guides and FIG. 5B shows the cut performance of the MAD2019 nuclease and various scaffolds with two CREATE Fusion (CF) guides.

FIGS. 6A and 6B show the editing performance of CF MAD2017 nickase and various scaffolds with two CREATE Fusion (CF) guides.

FIGS. 7A and 7B show the cut performance of MAD2017 nuclease and various scaffolds with two CREATE Fusion (CF) guides for cutting activity.

FIG. 8A shows GFP to BFP editing in HEK293T cells comprising an integrated GFP locus using the MAD2017 and MAD2019 nickases. FIG. 8B shows GFP to GFP-cut activity in HEK293T cells with the MAD2017 and MAD2019 nucleases. It should be understood that the drawings are not necessarily to scale.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities. Moreover, all of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis and hybridization and ligation of polynucleotides. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach. Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry(4th Ed.) W. H. Freeman, New York N.Y.; Gait, “Oligonucleotide Synthesis: A Practical Approach” 1984, IRL Press, London: Nelson and Cox (2000), Lehninger. Principles of Biochemistry 3^{rd} Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology(Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy. National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in. e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it, is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may he used in connection with the presently described invention.

Where a range of values is provided, it, is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-S′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and-for some components-translated in an appropriate host cell.

The terms “CREATE fusion enzyme” or the terms “nickase fusion” or “nickase fusion enzyme” refer to a nucleic acid-guided nickase fused to a reverse transcriptase where the fused enzyme both binds and nicks a target sequence in a sequence-specific manner and is capable of utilizing a repair template to incorporate nucleotides into the target sequence at the site of the nick.

The terms “editing cassette”, “CREATE cassette”, “CREATE editing cassette”, “CREATE fusion editing cassette” or “CF editing cassette” refer to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclcasc.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Nucleic acid-guided editing components” refers to one, some, or all of a nucleic acid-guided nuclease or nickase fusion enzyme, a guide nucleic acid and a repair template.

A “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible.

As used herein the terms “repair template” or “donor nucleic acid” or “donor DNA” or “homology arm” or “HA” or “homology region” or “HR” refer to 1) nucleic acid that is designed to introduce a DNA sequence modification (insertion, deletion, substitution) into a locus by homologous recombination using nucleic acid-guided nucleases, or 2) a nucleic acid that serves as a template (including a desired edit) to be incorporated into target DNA by a reverse transcriptase portion of a nickase fusion enzyme in a CREATE fusion (CF) editing system. For homology-directed repair, the repair template must have sufficient homology to the regions flanking the “cut site” or the site to be edited in the genomic target sequence. For template-directed repair, the repair template has homology to the genomic target sequence except at the position of the desired edit although synonymous edits may be present in the homologous (e.g., non-edit) regions. The length of the repair template(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

The terms “target genomic DNA sequence”, “target sequence”, or “genomic target locus” and the like refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The terms “transformation”, “transfection” and “transduction” are used interchangeably herein to refer to the process of introducing exogenous DNA into cells.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In some embodiments, a coding sequence for a nucleic acid-guided nuclease is provided in a vector, referred to as an “engine vector.” In some embodiments, the editing cassette may be provided in a vector, referred to as an “editing vector.” In some embodiments, the coding sequence for the nucleic acid-guided nuclease and the editing cassette are provided in the same vector.

### Nucleic Acid-Guided Nuclease and Nickase Editing

The nucleic acid-guided nickases described herein are employed to allow one to perform nucleic acid nuclease-directed genome editing to introduce desired edits to a population of live mammalian cells. The nucleic acid-guided nickases described herein have been derived from nucleic acid-guided nucleases which were engineered to create a nick as opposed to a double-strand break. In addition to the nickases, gRNA scaffold (sgRNA) sequences have been identified to be used in a nucleic acid-guided nickase CF editing system with the engineered nickases to improve editing efficiency.

Generally, a nucleic acid-guided nuclease or nickase fusion enzyme complexed with an appropriate synthetic guide nucleic acid in a cell can cut the genome of the cell at a desired location. The guide nucleic acid helps the nucleic acid-guided nuclease or nickase fusion enzyme recognize and cut the DNA at a specific target sequence. By manipulating the nucleotide sequence of the guide nucleic acid, the nucleic acid-guided nuclease or nickase fusion enzyme may be programmed to target any DNA sequence for cleavage as long as an appropriate protospacer adjacent motif (PAM) is nearby. In certain aspects, the nucleic acid-guided nuclease system or nucleic acid-guided nickase fusion editing system (i.e., CF editing system) may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion enzyme.

In general, a guide nucleic acid (e.g., gRNA) complexes with a compatible nucleic acid-guided nuclease or nickase fusion enzyme and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. Preferably and typically, the guide nucleic acid comprises RNA and the gRNA is encoded by a DNA sequence on an editing cassette along with the coding sequence for a repair template. Covalently linking the gRNA and repair template allows one to scale up the number of edits that can be made in a population of cells tremendously. Methods and compositions for designing and synthesizing editing cassettes (e.g., CREATE cassettes) are described in U.S. Pat. Nos. 10,240,167; 10,266,849; 9,982,278; 10,351,877; 10,364,442; 10,435,715; 10,669,559; 10,711,284; 10,731,180, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion enzyme to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or gRNA/nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, in the case of mammalian cells the target sequence is typically a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA). The proto-spacer mutation (PAM) is a short nucleotide sequence recognized by the gRNA/nuclease complex. The precise preferred PAM sequence and length requirements for different nucleic acid-guided nucleases or nickase fusions vary; however, PAMs typically are 2-10 base-pair sequences adjacent or in proximity to the target sequence and, depending on the nuclease or nickase, can be 5′ or 3′ to the target sequence.

In most embodiments, genome editing of a cellular target sequence both introduces a desired DNA change (i.e., the desired edit) to a cellular target sequence, e.g., the genomic DNA of a cell, and removes, mutates, or renders inactive a proto-spacer/spacer mutation (PAM) region in the cellular target sequence (e.g., thereby rendering the target site immune to further nuclease binding). Rendering the PAM and/or spacer at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM or spacer can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM or spacer alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion enzyme can be codon optimized for expression in particular cell types, such as bacterial, yeast, and, here, mammalian cells. The choice of the nucleic acid-guided nuclease or nickase fusion enzyme to be employed depends on many factors, such as what type of edit is to be made in the target sequence and whether an appropriate PAM is located close to the desired target sequence. Nucleic acid-guided nucleases (i.e., CRISPR enzymes) of use in the methods described herein include but are not limited to Cas 9, Cas 12/CpfI, MAD2, or MAD7, MAD 2007 or other MADzymes and MADzyme systems (see U.S. Pat. Nos. 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occurring MADzymes). Nickase fusion enzymes typically comprise a CRISPR nucleic acid-guided nuclease engineered to cut one DNA strand in a target DNA rather than making a double-stranded cut, and the nickase portion is fused to a reverse transcriptase. For more information on nickases and nickase fusion editing see U.S. Pat. No. 10,689,669 and U.S. Ser. Nos. 16/740,418; 16/740,420 and 16/740,421, all of which were filed 11 Jan. 2020. A coding sequence for a desired nuclease or nickase fusion may be on an “engine vector” along with other desired sequences such as a selective marker or may be transfected into a cell as a protein or ribonucleoprotein (“RNP”) complex.

Another component of the nucleic acid-guided nuclease or nickase fusion system is the repair template comprising homology to the cellular target sequence. In some exemplary embodiments, the repair template is in the same editing cassette as (e.g., is covalently-linked to) the guide nucleic acid and typically is under the control of the same promoter as the gRNA (that is, a single promoter driving the transcription of both the editing gRNA and the repair template). The repair template is designed to serve as a template for homologous recombination with a cellular target sequence cleaved by a nucleic acid-guided nuclease or serve as the template for template-directed repair via a nickase fusion, as a part of the gRNA/nuclease complex. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Pat. No. 10,711,284.

In certain preferred aspects, the repair template can be provided as an oligonucleotide of between 20-300 nucleotides, more preferably between 50-250 nucleotides. As described infra, the repair template comprises a region that is complementary to a portion of the cellular target sequence. When optimally aligned, the repair template overlaps with (is complementary to) the cellular target sequence by, e.g., about as few as 4 (in the case of nickase fusions) and as many as 20, 25, 30, 35, 40, 50, 60, 70, 80, 90 or more nucleotides (in the case of nucleases). The repair template comprises a region complementary to the cellular target sequence flanking the edit locus or difference between the repair template and the cellular target sequence. The desired edit may comprise an insertion, deletion, modification, or any combination thereof compared to the cellular target sequence.

As described in relation to the gRNA, the repair template may be provided as part of a rationally-designed editing cassette along with a promoter to drive transcription of both the gRNA and repair template. As described below, the editing cassette may be provided as a linear editing cassette, or the editing cassette may be inserted into an editing vector. Moreover, there may be more than one, e.g., two, three, four, or more editing gRNA/repair template pairs rationally-designed editing cassettes linked to one another in a linear “compound cassette” or inserted into an editing vector; alternatively, a single rationally-designed editing cassette may comprise two to several editing gRNA/repair template pairs, where each editing gRNA is under the control of separate different promoters, separate promoters, or where all gRNAs/repair template pairs are under the control of a single promoter. In some embodiments the promoter driving transcription of the editing gRNA and the repair template (or driving more than one editing gRNA/repair template pair) is an inducible promoter. In many if not most embodiments of the compositions, methods, modules and instruments described herein, the editing cassettes make up a collection or library editing of gRNAs and of repair templates representing, e.g., gene-wide or genome-wide libraries of editing gRNAs and repair templates.

In addition to the repair template, the editing cassettes comprise one or more primer binding sites to allow for PCR amplification of the editing cassettes. The primer binding sites are used to amplify the editing cassette by using oligonucleotide primers, and may be biotinylated or otherwise labeled. In addition, the editing cassette may comprise a barcode. A barcode is a unique DNA sequence that corresponds to the repair template sequence such that the barcode serves as a proxy to identify the edit made to the corresponding cellular target sequence. The barcode typically comprises four or more nucleotides. Also, in preferred embodiments, an editing cassette or editing vector or engine vector further comprises one or more nuclear localization sequences (NLSs), such as about or more than about 1, 2, 3, 4, 5, 6, 7, 8, 9, 10, or more NLSs.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1: Exemplary Workflow Overview

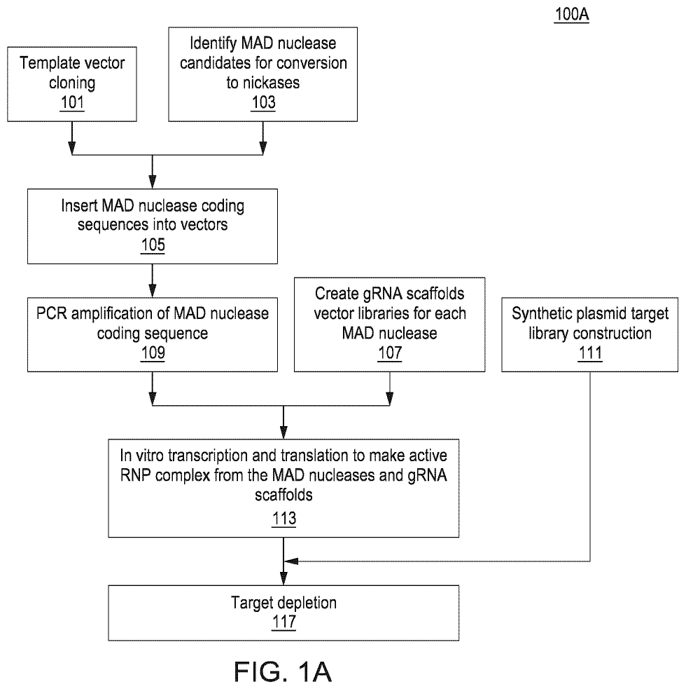

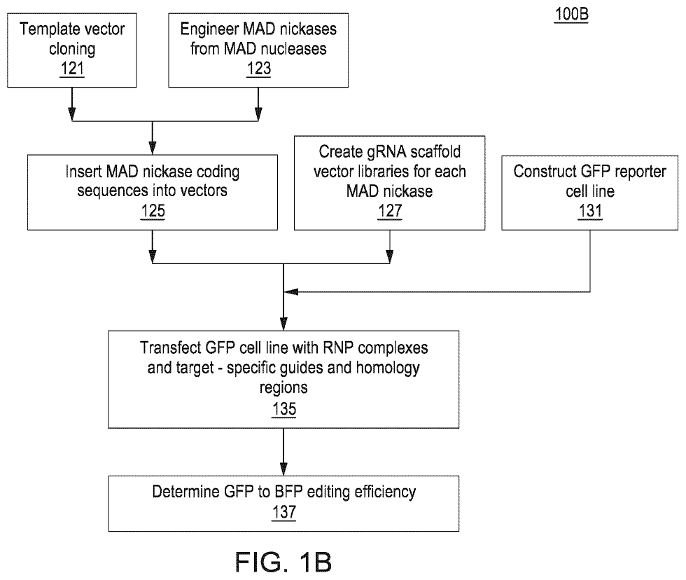

The disclosed MAD nucleic acid-guided nuclease and nickase fusion gRNA scaffolds were identified by the methods depicted in FIGS. 1A and 1B. The gRNA scaffolds form a part of a nucleic acid-guided nickase system for CF editing in cells. FIG. 1A shows an exemplary workflow 100A for screening MAD nucleic acid-guided nuclease and nickase scaffolds by determining cut activity using target depletion. In a first step 103, identified MAD nucleases identified as candidate for engineering. In parallel, a template vector is cloned 101. The coding sequences for the nucleic acid-guided nucleases are inserted into the template vector 105 and the nuclease sequences are amplified by PCR 109. Once the coding sequences for the nuclease are amplified 109, the native CRISPR repeat and tracrRNA for the nucleic acid-guided nuclease are used to construct variations on the gRNA scaffold structure 107 and are inserted into vector backbones. The nucleic acid-guided nucleases and sRNA scaffolds are transcribed, translated and combined to make active ribonucleoprotein (RNP) complexes 113. In parallel, synthetic targets were constructed 111 on which to test the RNP complexes for target depletion 117.

FIG. 1B shows an exemplary workflow 100B for screening of MAD nucleic acid-guided nickase scaffolds. In a first step 123, MAD nucleic acid-guided nucleases are identified as candidates for engineering nucleic acid-guided nickases. In parallel, a template vector is cloned 121. The coding sequences for the nucleic acid-guided nickases are inserted into the template vector 125 and the nuclease sequences are amplified by PCR. Once the coding sequences for the nickase are amplified, the native CRISPR repeat and tracrRNA for the nucleic acid-guided nuclease upon which the nickase is based are used to construct variations on the gRNA scaffold structure 127 and are inserted into vector backbones. The nucleic acid-guided nickases and gRNA scaffolds are transcribed, translated and combined to make active ribonucleoprotein (RNP) complexes. In parallel, a GFP reporter cell line is constructed 131. At step 135, the GFP reporter cell line is transfected with the RNP complexes and target-specific guides and homology regions. Finally, GFP to BFP editing efficiency 137 is determined.

Table 1 shows the amino acid sequences for the MAD2019 and MAD2017 nucleases on which the nickases are based (SEQ ID NO:1 and SEQ ID NO:2, respectively), as well as two nickases derived from each of these nucleases; namely, MAD2019-H848A (SEQ ID NO:3); MAD2019-N871A (SEQ ID NO:4); MAD2017-H847A (SEQ ID NO:5); and MAD2017-N870A (SEQ ID NO:6).

TABLE 1

| Sequence | | |
|---|---|---|
| Description | Derived from | Amino Acid Sequence |
| MAD2019 | Streptococcus | MTKPYSIGLDIGTNSVGWAVITDDYKVPSKKMKVLGNTSKKYIKKNLLGALLFDSGITAEGRRL |
| nuclease | sp. (firmicutes) | KRTARRRYTRRRNRILYLQEIFSTEMATLDDAFFQRLDDSFLVPDDKRDSKYPIFGNLVEEKAY |
| | | HDEFPTIYHLRKYLADSTKKADLRLVYLALAHMIKYRGHFLIEGEFNSKNNDIQKNFQDFLDTY |
| | | NAIFESDLSLENSKQLEEIVKDKISKLEKKDRILKLFPGEKNSGIFSEFLKLIVGNQADFKKYFNLD |
| | | EKASLHFSKESYDEDLETLLGYIGDDYSDVFLKAKKLYDAILLSGILTVTDNGTETPLSSAMIMRY |
| | | KEHEEDLGLLKAYIRNISLKTYNEVFNDDTKNGYAGYIDGKTNQEDFYVYLKKLLAKFEGADYF |
| | | LEKIDREDFLRKQRTFDNGSIPYQIHLQEMRAILDKQAKFYPFLAKNKERIEKILTFRIPYYVGPL |
| | | ARGNSDFAWSIRKRNEKITPWNFEDVIDKESSAEAFINRMTSFDLYLPEEKVLPKHSLLYETFT |
| | | VYNELTKVRFIAEGMSDYQFLDSKQKKDIVRLYFKGKRKVKVTDKDIIEYLHAIDGYDGIELKGI |
| | | EKQFNSSLSTYHDLLNIINDKEFLDDSSNEAIIEEIIHTLTIFEDREMIKQRLSKFENIFDKSVLKKLS |
| | | RRHYTGWGKLSAKLINGIRDEKSGNTILDYLIDDGISNRNFMQLIHDDALSFKKKIQKAQIIGD |
| | | KDKDNIKEVVKSLPGSPAIKKGILQSIKIVDELVKVMGRKPESIVVEMARENQYTNQGKSNSQ |
| | | QRLKRLEESLEELGSKILKENIPAKLSKIDNNSLQNDRLYLYYLQNGKDMYTGDDLDIDRLSNYD |
| | | IDHIIPQAFLKDNSIDNKVLVSSASNRGKSDDVPSLEVVKKRKTLWYQLLKSKLISQRKFDNLTK |
| | | AERGGLSPEDKAGFIQRQLVETRQITKHVARLLDEKFNNKKDENNRAVRTVKIITLKSTLVSQF |
| | | RKDFELYKVREINDFHHAHDAYLNAVVASALLKKYPKLEPEFVYGDYPKYNSFRERKSATEKVY |
| | | FYSNIMNIFKKSISLADGRVIERPLIEVNEETGESVWNKESDLATVRRVLSYPQVNVVKKVEVQ |
| | | SGGFSKELVQPHGNSDKLIPRKTKKMIWDTKKYGGFDSPIVAYSVLVMAEREKGKSKKLKPVK |
| | | ELVRITIMEKESFKENTIDFLERRGLRNIQDENIILLPKFSLFELENGRRRLLASAKELQKGNEFILP |
| | | NKLVKLLYHAKNIHNTLEPEHLEYVESHRADFGKILDVVSVFSEKYILAEAKLEKIKEIYRKNMNT |
| | | EIHEMATAFINLLTFTSIGAPATFKFFGHNIERKRYSSVAEILNATLIHQSVTGLYETRIDLGKLGE |
| | | D |
| | | [SEQ ID NO: 1] |
| MAD2017 | Streptococcus | MKKPYSIGLDIGTNSVGWAVITDDYKVPAKKMKVLGNTDKKYIKKNLLGALLFDSGETAEVTR |
| nuclease | sp. (firmicutes) | LKRTARRRYTRRKNRLRYLQEIFAKEMTKVDESFFQRLEESFLTDDDKTFDSHPIFGNKAEEDA |
| | | YHQKFPTIYHLRKYLADSQEKADLRLVYLALAHMIKYRGHFLIEGELNAENTDVQKLFNVFVET |
| | | YDKIVDESHLSEIEVDASSILTEKVSKSRRLENLIKQYPTEKKNTLFGNLIALALGLQPNFKTNFKL |
| | | SEDAKLQFSKDTYEEDLEELLGKVGDDYADLFISAKNLYDAILLSGILTVDDNSTKAPLSASMIK |
| | | RYVEHHEDLEKLKEFIKINKLKLYHDIFKDKTKNGYAGYIDNGVKQDEFYKYLKTILTKIDDSDYF |
| | | LDKIERDDFLRKQRTFDNGSIPHQIHLQEMHSILRRQGEYYPFLKENQAKIEKILTFRIPYYVGPL |
| | | ARKDSRFAWANYHSDEPITPWNFDEVVDKEKSAEKFITRMTLNDLYLPEEKVLPKHSHVYETF |
| | | TVYNELTKIKYVNEQGESFFFDANMKQEIFDHVFKENRKVTKAKLLSYLNNEFEEFRINDLIGL |
| | | DKDSKSFNASLGTYHDLKKILDKSFLDDKTNEQIIEDIVLTLTLFEDRDMIHERLQKYSDFFTSQ |
| | | QLKKLERRHYTGWGRLSYKLINGIRNKENNKTILDFLIDDGHANRNFMQLINDESLSFKTIIQE |
| | | AQVVGDVDDIEAVVHDLPGSPAIKKGILQSVKIVDELVKVMGDNPDNIVIEMARENQTTGYG |
| | | RNKSNQRLKRLQDSLKEFGSDILSKKKPSYVDSKVENSHLQNDRLFLYYIQNGKDMYTGEELDI |
| | | DRLSDYDIDHIIPQAFIKDNSIDNKVLTSSAKNRGKSDDVPSIEIVRNRRSYWYKLYKSGLISKRK |
| | | FDNLTKAERGGLTEADKAGFIKRQLVETRQITKHVAQILDARFNTKRDENDKVIRDVKVITLKS |
| | | NLVSQFRKEFKFYKVREINDYHHANDAYLNAVVGTALLKKYPKLTPEFVYGEYKKYDVRKLIAK |
| | | SSDDYSEMGKATAKYFFYSNLMNFFKTEVKYADGRVFERPDIETNADGEVVWNKQKDFDIV |
| | | RKVLSYPQVNIVKKVEAQTGGFSKESILSKGDSDKLIPRKTKKVYWNTKKYGGFDSPTVAYSVL |
| | | VVADIEKGKAKKLKTVKELVGISIMERSFFEENPVSFLEKKGYHNVQEDKLIKLPKYSLFEFEGG |
| | | RRRLLASATELQKGNEVMLPAHLVELLYHAHRIDSFNSTEHLKYVSEHKKEFEKVLSCVENFSN |
| | | LYVDVEKNLSKVRAAAESMTNFSLEEISASFINLLTLTALGAPADFNFLGEKIPRKRYTSTKECLS |
| | | ATLIHQSVTGLYETRIDLSKLGEE |
| | | [SEQ ID NO: 2] |
| MAD2019 | Streptococcus | MTKPYSIGLDIGTNSVGWAVITDDYKVPSKKMKVLGNTSKKYIKKNLLGALLFDSGITAEGRRL |
| Nickase | sp. (firmicutes) | KRTARRRYTRRRNRILYLQEIFSTEMATLDDAFFQRLDDSFLVPDDKRDSKYPIFGNLVEEKAY |
| H848A | then | HDEFPTIYHLRKYLADSTKKADLRLVYLALAHMIKYRGHFLIEGEFNSKNNDIQKNFQDFLDTY |
| | engineered | NAIFESDLSLENSKQLEEIVKDKISKLEKKDRILKLFPGEKNSGIFSEFLKLIVGNQADFKKYFNLD |
| | | EKASLHFSKESYDEDLETLLGYIGDDYSDVFLKAKKLYDAILLSGILTVTDNGTETPLSSAMIMRY |
| | | KEHEEDLGLLKAYIRNISLKTYNEVFNDDTKNGYAGYIDGKTNQEDFYVYLKKLLAKFEGADYF |
| | | LEKIDREDFLRKQRTFDNGSIPYQIHLQEMRAILDKQAKFYPFLAKNKERIEKILTFRIPYYVGPL |
| | | ARGNSDFAWSIRKRNEKITPWNFEDVIDKESSAEAFINRMTSFDLYLPEEKVLPKHSLLYETFT |
| | | VYNELTKVRFIAEGMSDYQFLDSKQKKDIVRLYFKGKRKVKVTDKDIIEYLHAIDGYDGIELKGI |
| | | EKQFNSSLSTYHDLLNIINDKEFLDDSSNEAIIEEIIHTLTIFEDREMIKQRLSKFENIFDKSVLKKLS |
| | | RRHYTGWGKLSAKLINGIRDEKSGNTILDYLIDDGISNRNFMQLIHDDALSFKKKIQKAQIIGD |
| | | KDKDNIKEVVKSLPGSPAIKKGILQSIKIVDELVKVMGRKPESIVVEMARENQYTNQGKSNSQ |
| | | QRLKRLEESLEELGSKILKENIPAKLSKIDNNSLQNDRLYLYYLQNGKDMYTGDDLDIDRLSNYD |
| | | IDAIIPQAFLKDNSIDNKVLVSSASNRGKSDDVPSLEVVKKRKTLWYQLLKSKLISQRKFDNLTK |
| | | AERGGLSPEDKAGFIQRQLVETRQITKHVARLLDEKFNNKKDENNRAVRTVKIITLKSTLVSQF |
| | | RKDFELYKVREINDFHHANDAYLNAVVASALLKKYPKLEPEFVYGDYPKYNSFRERKSATEKVY |
| | | FYSNIMNIFKKSISLADGRVIERPLIEVNEETGESVWNKESDLATVRRVLSYPQVNVVKKVEVQ |
| | | SGGFSKELVQPHGNSDKLIPRKTKKMIWDTKKYGGFDSPIVAYSVLVMAEREKGKSKKLKPVK |
| | | ELVRITIMEKESFKENTIDFLERRGLRNIQDENIILLPKFSLFELENGRRRLLASAKELQKGNEFILP |
| | | NKLVKLLYHAKNIHNTLEPEHLEYVESHRADFGKILDVVSVFSEKYILAEAKLEKIKEIYRKNMNT |
| | | EIHEMATAFINLLTFTSIGAPATFKFFGHNIERKRYSSVAEILNATLIHQSVTGLYETRIDLGKLGE |
| | | D |
| | | [SEQ ID NO: 3] |
| MAD2019 | Streptococcus | MTKPYSIGLDIGTNSVGWAVITDDYKVPSKKMKVLGNTSKKYIKKNLLGALLFDSGITAEGRRL |
| Nickase | sp. (firmicutes) | KRTARRRYTRRRNRILYLQEIFSTEMATLDDAFFQRLDDSFLVPDDKRDSKYPIFGNLVEEKAY |
| N871A | then | HDEFPTIYHLRKYLADSTKKADLRLVYLALAHMIKYRGHFLIEGEFNSKNNDIQKNFQDFLDTY |
| | engineered) | NAIFESDLSLENSKQLEEIVKDKISKLEKKDRILKLFPGEKNSGIFSEFLKLIVGNQADFKKYFNLD |
| | | EKASLHFSKESYDEDLETLLGYIGDDYSDVFLKAKKLYDAILLSGILTVTDNGTETPLSSAMIMRY |
| | | KEHEEDLGLLKAYIRNISLKTYNEVFNDDTKNGYAGYIDGKTNQEDFYVYLKKLLAKFEGADYF |
| | | LEKIDREDFLRKQRTFDNGSIPYQIHLQEMRAILDKQAKFYPFLAKNKERIEKILTFRIPYYVGPL |
| | | ARGNSDFAWSIRKRNEKITPWNFEDVIDKESSAEAFINRMTSFDLYLPEEKVLPKHSLLYETFT |
| | | VYNELTKVRFIAEGMSDYQFLDSKQKKDIVRLYFKGKRKVKVTDKDIIEYLHAIDGYDGIELKGI |
| | | EKQFNSSLSTYHDLLNIINDKEFLDDSSNEAIIEEIIHTLTIFEDREMIKQRLSKFENIFDKSVLKKLS |
| | | RRHYTGWGKLSAKLINGIRDEKSGNTILDYLIDDGISNRNFMQLIHDDALSFKKKIQKAQIIGD |
| | | KDKDNIKEVVKSLPGSPAIKKGILQSIKIVDELVKVMGRKPESIVVEMARENQYTNQGKSNSQ |
| | | QRLKRLEESLEELGSKILKENIPAKLSKIDNNSLQNDRLYLYYLQNGKDMYTGDDLDIDRLSNYD |
| | | IDHIIPQAFLKDNSIDNKVLVSSASARGKSDDVPSLEVVKKRKTLWYQLLKSKLISQRKFDNLTK |
| | | AERGGLSPEDKAGFIQRQLVETRQITKHVARLLDEKFNNKKDENNRAVRTVKIITLKSTLVSQF |
| | | RKDFELYKVREINDFHHANDAYLNAVVASALLKKYPKLEPEFVYGDYPKYNSFRERKSATEKVY |
| | | FYSNIMNIFKKSISLADGRVIERPLIEVNEETGESVWNKESDLATVRRVLSYPQVNVVKKVEVQ |
| | | SGGFSKELVQPHGNSDKLIPRKTKKMIWDTKKYGGFDSPIVAYSVLVMAEREKGKSKKLKPVK |
| | | ELVRITIMEKESFKENTIDFLERRGLRNIQDENIILLPKFSLFELENGRRRLLASAKELQKGNEFILP |
| | | NKLVKLLYHAKNIHNTLEPEHLEYVESHRADFGKILDVVSVFSEKYILAEAKLEKIKEIYRKNMNT |
| | | EIHEMATAFINLLTFTSIGAPATFKFFGHNIERKRYSSVAEILNATLIHQSVTGLYETRIDLGKLGE |
| | | D |
| | | [SEQ ID NO: 4] |
| MAD2017 | Streptococcus | MKKPYSIGLDIGTNSVGWAVITDDYKVPAKKMKVLGNTDKKYIKKNLLGALLFDSGETAEVTR |
| nickase | sp. (firmicutes) | LKRTARRRYTRRKNRLRYLQEIFAKEMTKVDESFFQRLEESFLTDDDKTFDSHPIFGNKAEEDA |
| H847A | then | YHQKFPTIYHLRKYLADSQEKADLRLVYLALAHMIKYRGHFLIEGELNAENTDVQKLFNVFVET |
| | engineered) | YDKIVDESHLSEIEVDASSILTEKVSKSRRLENLIKQYPTEKKNTLFGNLIALALGLQPNFKTNFKL |
| | | SEDAKLQFSKDTYEEDLEELLGKVGDDYADLFISAKNLYDAILLSGILTVDDNSTKAPLSASMIK |
| | | RYVEHHEDLEKLKEFIKINKLKLYHDIFKDKTKNGYAGYIDNGVKQDEFYKYLKTILTKIDDSDYF |
| | | LDKIERDDFLRKQRTFDNGSIPHQIHLQEMHSILRRQGEYYPFLKENQAKIEKILTFRIPYYVGPL |
| | | ARKDSRFAWANYHSDEPITPWNFDEVVDKEKSAEKFITRMTLNDLYLPEEKVLPKHSHVYETF |
| | | TVYNELTKIKYVNEQGESFFFDANMKQEIFDHVFKENRKVTKAKLLSYLNNEFEEFRINDLIGL |
| | | DKDSKSFNASLGTYHDLKKILDKSFLDDKTNEQIIEDIVLTLTLFEDRDMIHERLQKYSDFFTSQ |
| | | QLKKLERRHYTGWGRLSYKLINGIRNKENNKTILDFLIDDGHANRNFMQLINDESLSFKTIIQE |
| | | AQVVGDVDDIEAVVHDLPGSPAIKKGILQSVKIVDELVKVMGDNPDNIVIEMARENQTTGYG |
| | | RNKSNQRLKRLQDSLKEFGSDILSKKKPSYVDSKVENSHLQNDRLFLYYIQNGKDMYTGEELDI |
| | | DRLSDYDIDAIIPQAFIKDNSIDNKVLTSSAKNRGKSDDVPSIEIVRNRRSYWYKLYKSGLISKRK |
| | | FDNLTKAERGGLTEADKAGFIKRQLVETRQITKHVAQILDARFNTKRDENDKVIRDVKVITLKS |
| | | NLVSQFRKEFKFYKVREINDYHHAHDAYLNAVVGTALLKKYPKLTPEFVYGEYKKYDVRKLIAK |
| | | SSDDYSEMGKATAKYFFYSNLMNFFKTEVKYADGRVFERPDIETNADGEVVWNKQKDFDIV |
| | | RKVLSYPQVNIVKKVEAQTGGFSKESILSKGDSDKLIPRKTKKVYWNTKKYGGFDSPTVAYSVL |
| | | VVADIEKGKAKKLKTVKELVGISIMERSFFEENPVSFLEKKGYHNVQEDKLIKLPKYSLFEFEGG |
| | | RRRLLASATELQKGNEVMLPAHLVELLYHAHRIDSFNSTEHLKYVSEHKKEFEKVLSCVENFSN |
| | | LYVDVEKNLSKVRAAAESMTNFSLEEISASFINLLTLTALGAPADFNFLGEKIPRKRYTSTKECLS |
| | | ATLIHQSVTGLYETRIDLSKLGEE |
| | | [SEQ ID NO: 5] |
| MAD2017 | Streptococcus | MKKPYSIGLDIGTNSVGWAVITDDYKVPAKKMKVLGNTDKKYIKKNLLGALLFDSGETAEVTR |
| nickase | sp. (firmicutes) | LKRTARRRYTRRKNRLRYLQEIFAKEMTKVDESFFQRLEESFLTDDDKTFDSHPIFGNKAEEDA |
| N870A | then | YHQKFPTIYHLRKYLADSQEKADLRLVYLALAHMIKYRGHFLIEGELNAENTDVQKLFNVFVET |
| | engineered | YDKIVDESHLSEIEVDASSILTEKVSKSRRLENLIKQYPTEKKNTLFGNLIALALGLQPNFKTNFKL |
| | | SEDAKLQFSKDTYEEDLEELLGKVGDDYADLFISAKNLYDAILLSGILTVDDNSTKAPLSASMIK |
| | | RYVEHHEDLEKLKEFIKINKLKLYHDIFKDKTKNGYAGYIDNGVKQDEFYKYLKTILTKIDDSDYF |
| | | LDKIERDDFLRKQRTFDNGSIPHQIHLQEMHSILRRQGEYYPFLKENQAKIEKILTFRIPYYVGPL |
| | | ARKDSRFAWANYHSDEPITPWNFDEVVDKEKSAEKFITRMTLNDLYLPEEKVLPKHSHVYETF |
| | | TVYNELTKIKYVNEQGESFFFDANMKQEIFDHVFKENRKVTKAKLLSYLNNEFEEFRINDLIGL |
| | | DKDSKSFNASLGTYHDLKKILDKSFLDDKTNEQIIEDIVLTLTLFEDRDMIHERLQKYSDFFTSQ |
| | | QLKKLERRHYTGWGRLSYKLINGIRNKENNKTILDFLIDDGHANRNFMQLINDESLSFKTIIQE |
| | | AQVVGDVDDIEAVVHDLPGSPAIKKGILQSVKIVDELVKVMGDNPDNIVIEMARENQTTGYG |
| | | RNKSNQRLKRLQDSLKEFGSDILSKKKPSYVDSKVENSHLQNDRLFLYYIQNGKDMYTGEELDI |
| | | DRLSDYDIDHIIPQAFIKDNSIDNKVLTSSAKARGKSDDVPSIEIVRNRRSYWYKLYKSGLISKRK |
| | | FDNLTKAERGGLTEADKAGFIKRQLVETRQITKHVAQILDARFNTKRDENDKVIRDVKVITLKS |
| | | NLVSQFRKEFKFYKVREINDYHHAHDAYLNAVVGTALLKKYPKLTPEFVYGEYKKYDVRKLIAK |
| | | SSDDYSEMGKATAKYFFYSNLMNFFKTEVKYADGRVFERPDIETNADGEVVWNKQKDFDIV |
| | | RKVLSYPQVNIVKKVEAQTGGFSKESILSKGDSDKLIPRKTKKVYWNTKKYGGFDSPTVAYSVL |
| | | VVADIEKGKAKKLKTVKELVGISIMERSFFEENPVSFLEKKGYHNVQEDKLIKLPKYSLFEFEGG |
| | | RRRLLASATELQKGNEVMLPAHLVELLYHAHRIDSFNSTEHLKYVSEHKKEFEKVLSCVENFSN |
| | | LYVDVEKNLSKVRAAAESMTNFSLEEISASFINLLTLTALGAPADFNFLGEKIPRKRYTSTKECLS |
| | | ATLIHQSVTGLYETRIDLSKLGEE |
| | | [SEQ ID NO: 6] |

### Example 2: Scaffold Optimization for the MAD2019 and MAD2017 Nickases

MAD2019 Nuclease: Three versions of gRNA scaffolds were designed using the MAD2019 nuclease native CRISPR repeat and tracr RNA sequences (corresponding to step 107 of FIG. 1A). The native CRISPR repeat and tracr RNA for the MAD2019 nuclease (SEQ ID NOs: 7 and 8, respectively), as well as the variant gRNA scaffolds (sgRNA) for MAD2019 (i.e., gRNA scaffold 2019v1 [SEQ ID NO:9]; gRNA scaffold 2019v2 [SEQ ID NO:10]; and gRNA scaffold 2019v3 [SEQ ID NO:11]) are shown in Table 2.

TABLE 2
First Round of Scaffold Optimization for MAD2019 Nickase

| Sequence Name | Sequence |
|---|---|
| Native CRISPR | 5′-GTTTTAGAGCTGTGTTGTTTCGAATGGTTCCAAAAC-3′ |
| repeat MAD2019 | [SEQ ID NO: 7] |
| Native tracr RNA | 5′-GGTTTGAAACCATTCGAAACAATACAGCAAAGTTAAAATAAGGCTA |
| MAD2019 | GTCCGTATACAACGTGAAAACACGTGGCACCGATTCGGTGC-3′ |
| | [SEQ ID NO: 8] |
| sgRNA2019v1 | 5′-GTTTTAGAGCTGTGTTGTTTCGAATGGTTCCAAAACGGTTTGAAACCAT |
| | TCGAAACAATACAGCAAAGTTAAAATAAGGCTAGTCCGTATACAACGTGA |
| | AAACACGTGGCACCGATTCGGTGC-3′ |
| | [SEQ ID NO: 9] |
| sgRNA 2019v2 | 5′-GTTTTAGAGCTGTGTTGTAAAAACAATACAGCAAAGTTAAAATAAGGCT |
| | AGTCCGTATACAACGTGAAAACACGTGGCACCGATTCGGTGC-3′ |
| | [SEQ ID NO: 10] |
| sgRNA 2019v3 | 5′-GTTTTAGAGCTGTGTTGTAAAAACAATACAGCAAGTTAAAATAAGGCTA |
| | GTCCGTATACAACGTGAAAACACGTGGCACCGATTCGGTGC-3′ |
| | [SEQ ID NO: 11] |

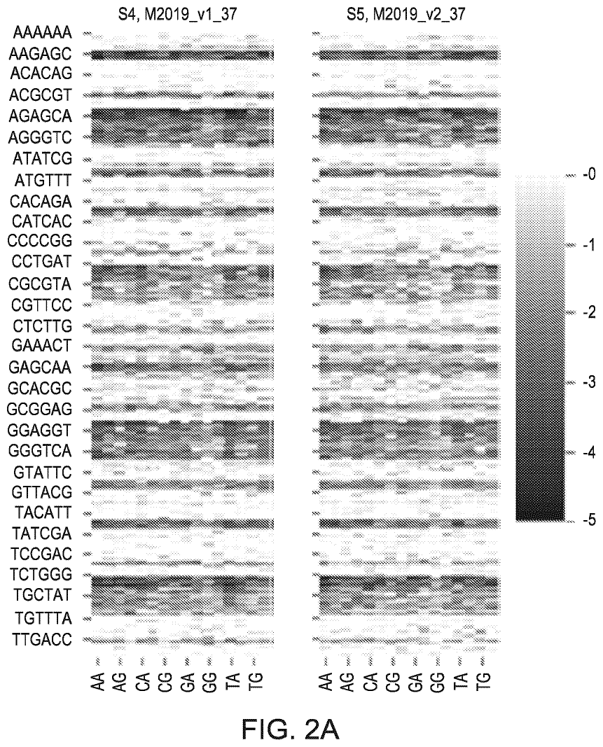

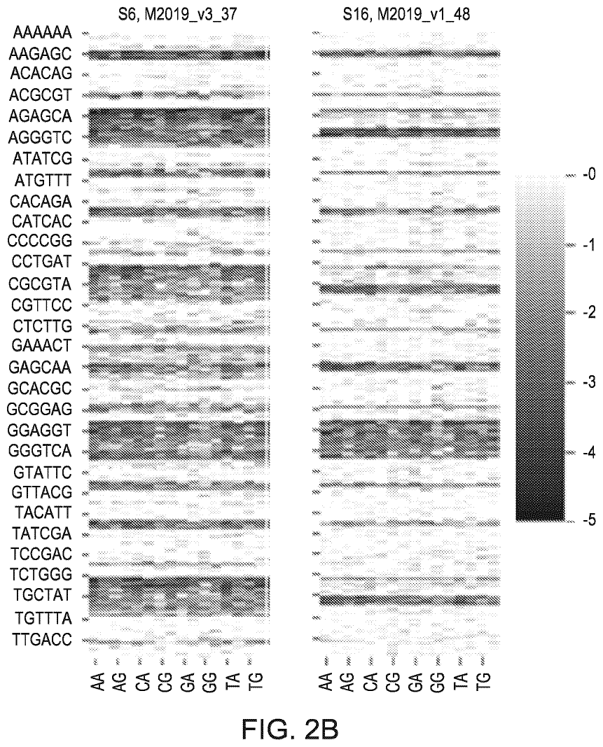

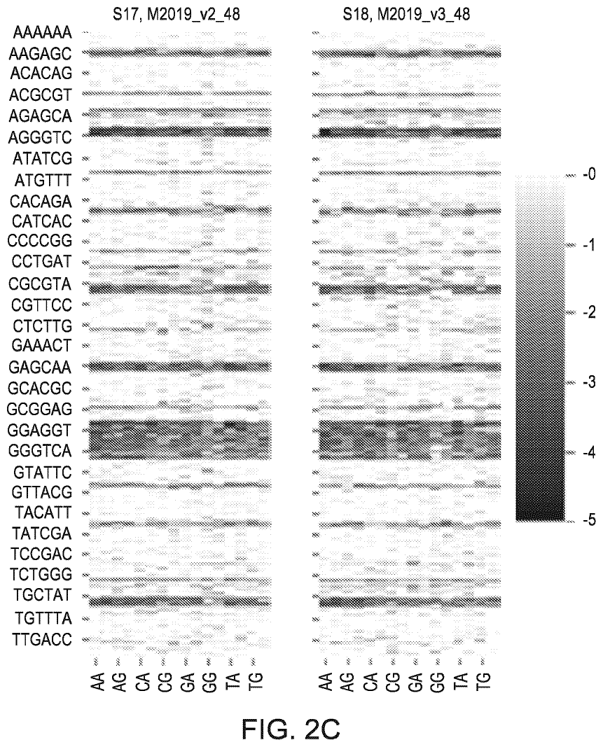

The MAD2019 nuclease and variant guide RNAs were produced in vitro to form RNP complexes (corresponding to step 113 of FIG. 1A) and the digestion patterns with a plasmid target, degenerate PAM sequences at two different temperatures (37° C. and 48° C.) were compared. The results are shown in FIGS. 2A-2C. There were no differences in performance in vitro between different variant sgRNAs used. An 8N degenerate PAM sequence was used in this assay. The Y-axis of FIGS. 2A-2C is for the first six nucleotides of the PAM and the two last nucleotides of the PAM are shown on the X-axis. A darker the color shows more depletion and higher activity.

MAD2017 Nuclease: Three versions of sgRNA scaffolds were designed using the MAD2017 nuclease native CRISPR repeat and tracr RNA sequences (corresponding to step 107 of FIG. 1A). The native CRISPR repeat and tracr RNA for the MAD2017 nuclease (SEQ ID NOs: 12 and 13, respectively), as well as the variant gRNA scaffolds for MAD2017 (i.e., gRNA scaffold2017v2 [SEQ ID NO:14]; gRNA scaffold2017v3 [SEQ ID NO:15]; and gRNA scaffold 2017v4 [SEQ ID NO:16]) are shown in Table 3.

TABLE 3
First Round of Scaffold Optimization for MAD2017 Nickase

| Sequence Name | Sequence |
|---|---|
| Native CRISPR | 5′-GTTTTAGAGCTGTGCTGTTTCGAATGGTTCCAAAAC-3′ |
| repeat MAD2017 | [SEQ ID NO: 12] |
| Native tracr RNA | 5′-TGTTGGAACTATTCGAAACAACACAGCGAGTTAAAATAAGGCTTT |
| MAD2017 | GTCCGTACACAACTTGTAAAAGGGGCACCCGATTCGGGTGCA-3′ |
| | [SEQ ID NO: 13] |
| sgRNA 2017v2 | 5′-GTTTTAGAGCTGTGCTGTTTCGAAAAATCGAAACAACACAGCGAGT |
| | TAAAATAAGGCTTTGTCCGTACACAACTTGTAAAAGGGGCACCCGATT |
| | CGGGTGC-3′ |
| | [SEQ ID NO: 14] |
| sgRNA 2017v3 | 5′-GTTTTAGAGCTGTGCTGTAAAAACAACACAGCGAGTTAAAATAAGG |
| | CTTTGTCCGTACACAACTTGTAAAAGGGGCACCCGATTCGGGTGC-3′ |
| | [SEQ ID NO: 15] |
| sgRNA 2017v4 | 5′-GTTTTAGAGCTGTGCAAACACAGCGAGTTAAAATAAGGCTTTGTCC |
| | GTACACAACTTGTAAAAGGGGCACCCGATTCGGGTGC-3′ |
| | [SEQ ID NO: 16] |

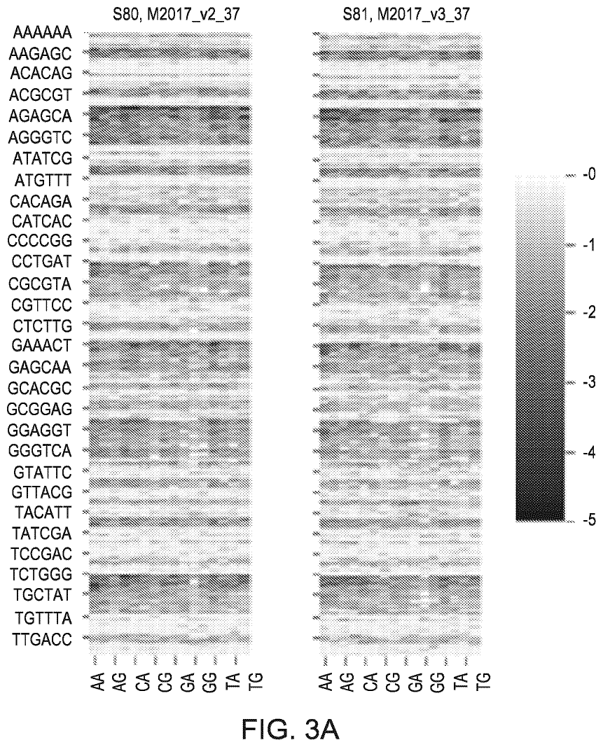

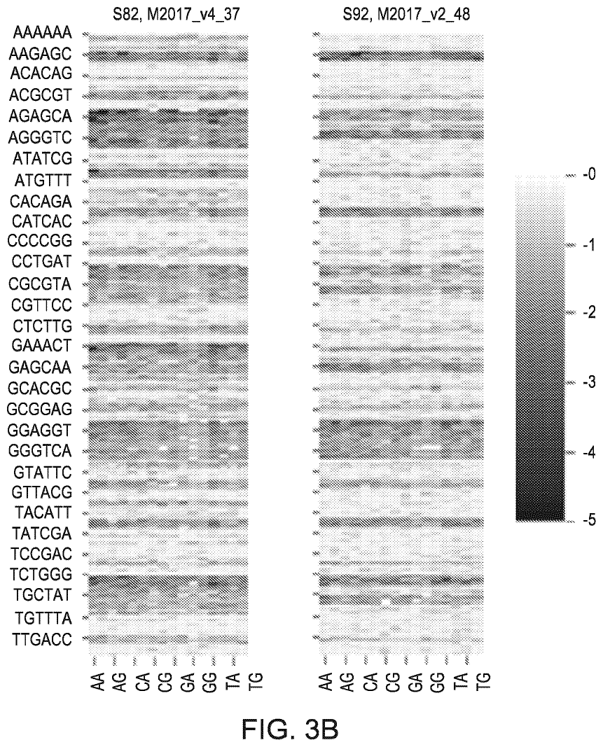

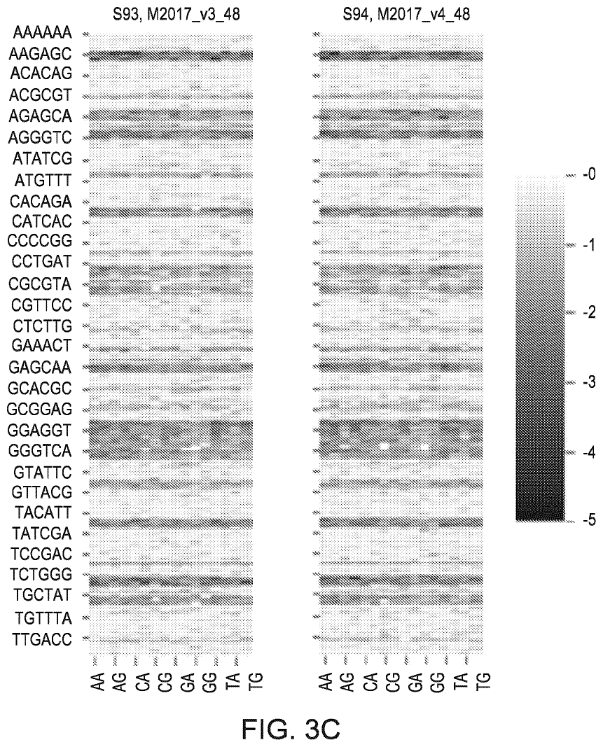

The MAD2017 nuclease and variant guide RNAs were produced in vitro to form RNP complexes (corresponding to step 113 of FIG. 1A) and the digestion patterns with a plasmid target, degenerate PAM sequences at two different temperatures (37° C. and 48° C.) were compared. The results are shown in FIGS. 3A-3C. There were no differences in performance in vitro between different variant sgRNAs used for the guide RNA production. An 8N degenerate PAM sequence was used in this assay. The Y-axis of FIGS. 3A-3C is for the first six nucleotides of the PAM and the two last nucleotides of the PAM are shown on the X-axis. A darker the color shows more depletion and higher activity.

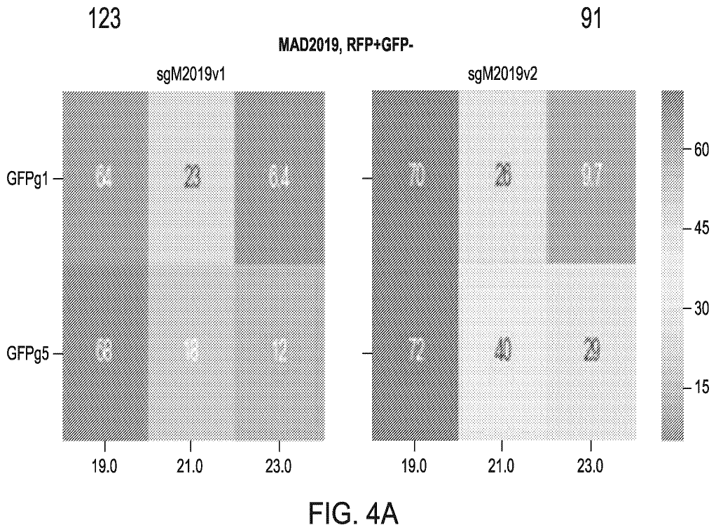

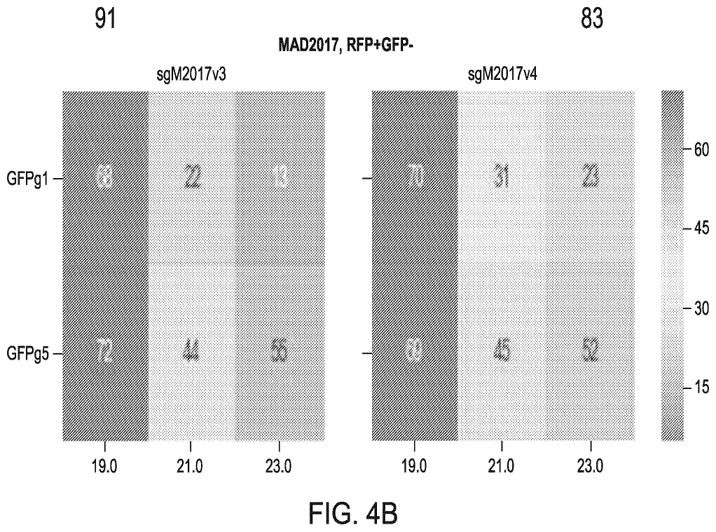

In vivo test of two scaffolds: A variant from the gRNA scaffolds listed in Tables 2 and 3 for each of MAD2019 and MAD2017 was used to test the effect on double strand break (DSB) formation activity on a GFP locus integrated in HEK293T cells. Three different guide lengths were tested (x-axis shown in FIGS. 4A and 4B), and two different guides (GFPg1 and GFPg5 in y-axis in FIGS. 4A and 4B) were used in different combinations. The sequences for GFPg1 and GFPg5 are shown in Table 4 below.

TABLE 4
Design of Full Guide: spacer + sgRNA scaffold + HR + G4

| Name of Guide | Guide Sequence | HR in CREATE fusion | G4 |
|---|---|---|---|
| GFPg1CF | GCTGAAGCACTGCACGC | ACCCTCAGCCACGGCGTGCAGT | ACTAACGGTGGTGG |
| | CGT [SEQ ID NO: 28] | GCTT [SEQ ID NO: 29] | TGG [SEQ ID NO: 30] |
| GFPg5CF | GGTGCTGCTTCATGTGGT | ACCCTCAGCCACGGCGTGCAGT | ACTAACGGTGGTGG |
| | CG [SEQ ID NO: 31] | GCTTCAGCCGCTATCCCGACCA | TGG [SEQ ID NO: 30] |
| | | CATGAAGCAG [SEQ ID NO: 32] | |

Two separate plasmids expressing either the MAD nuclease or a guide were transfected into the HEK293T cells with the integrated GFP locus. The coding sequences for the MAD nucleases were expressed under a CMV promoter cloned on a plasmid. The guides were produced from a separate plasmid expressed under the U6 promoter. The expression of the MAD nucleases was measured by an RFP signal produced as a T2A connected self-splicing protein fused to the carboxy terminus of the respective MAD nuclease. Double-strand break activity was measured from the population of HEK293T cells that retained RFP signal after transfection and expanded for 5-6 days. The results are shown in FIGS. 4A and 4B. For both the MAD2019 and MAD2017 nucleases, the 19-bp guides were the most efficient; and with this optimal guide length, there were no differences in cutting activity.

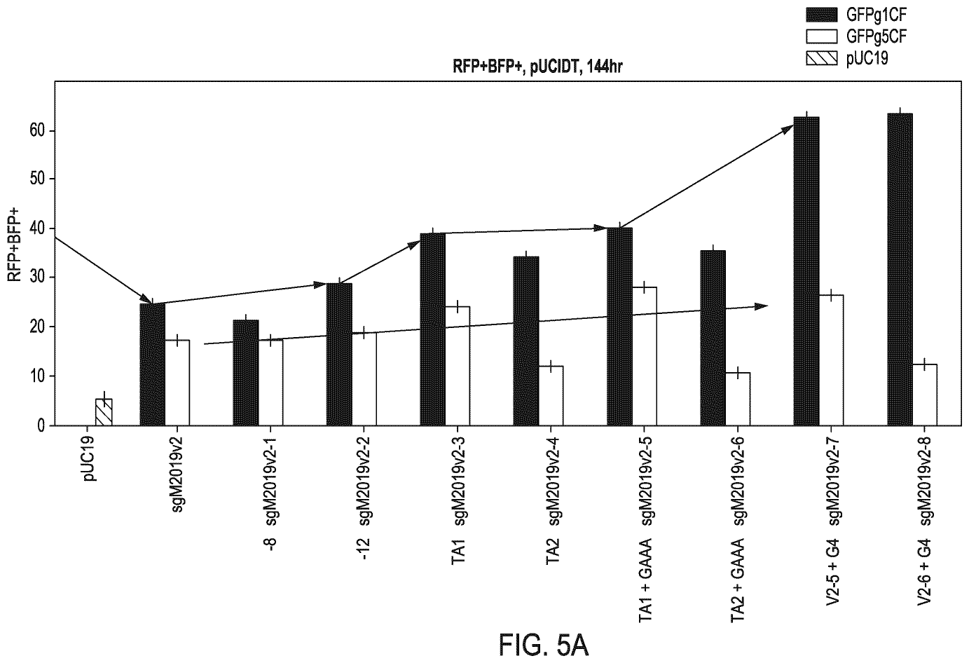

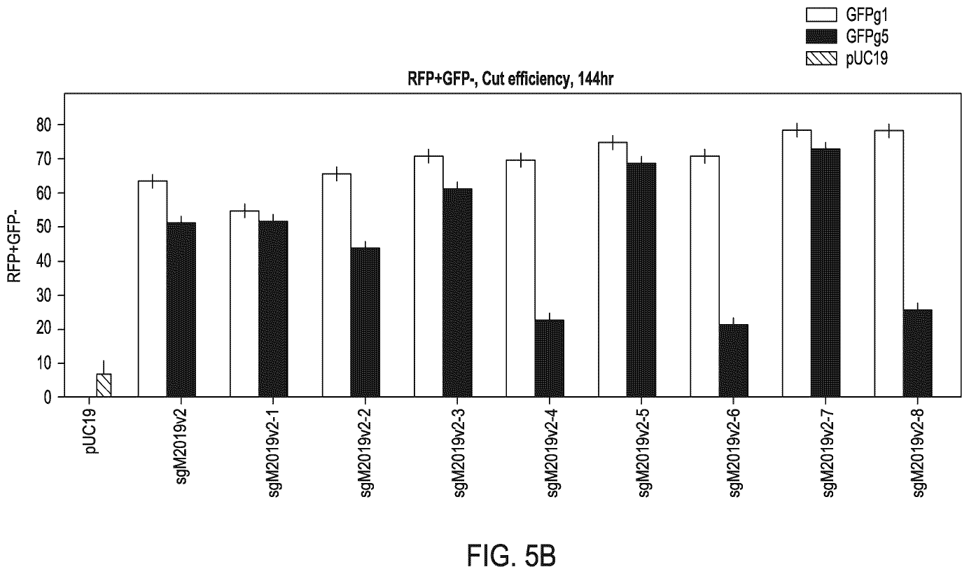

Further Optimization of seRNA scaffolds: The MAD2019v2 sequence was used to further optimize the sgRNA scaffold. The sequence for each of these variants is shown in Table 5. The full guide comprised from 5′ to 3′: spacer+sgRNA scaffold (Table 5)+CF (HR in CREATE FUSION column in Table 4)+G4 (if included in the scaffold design) (also in Table 4). The guides were expressed under a U6 promoter. Editing performance using the MAD2019 nickase fused to a reverse transcriptase was measured by performing in vivo editing on the GFP locus in HEK293T cells changing GFP to BFP (see method depicted in FIG. 1B). The BFP signal was measured from cells maintaining an RFP signal after transfection. The results are shown in FIGS. 5A and 5B. In FIG. 5A. performance for each gRNA scaffold with the two different CF guides (GFPg1CF (dark bars) and GFPg5CF (light bars)) was measured. Improvement of performance is noticeable with GFPg1CF.

Cutting performance for each sRNA scaffold design with GFPg1CF (light bar) and GFPg5CF (medium dark bar) is shown in FIG. 5B. Cutting performance was measured with wildtype MAD2019 nuclease using the same guide designs used for the CF editing measured with the results shown in FIG. 5A.

TABLE 5
Second Round of Optimization for MAD2019 Nickase

| Sequence Name | Sequence |
|---|---|
| sgRNA 2019v2-1 | GTTTTAGAGCTGTGGAAATACAGCAAAGTTAAAATAAGGCTAGTCCGTATACAACGTG |
| | AAAACACGTGGCACCGATTCGGTGC [SEQ ID NO: 17] |
| sgRNA 2019v2-2 | GTTTTAGAGCTGGAAACAGCAAAGTTAAAATAAGGCTAGTCCGTATACAACGTGAAA |
| | ACACGTGGCACCGATTCGGTGC [SEQ ID NO: 18] |
| sgRNA 2019v2-3 | GTTTAAGAGCTGGAAACAGCAAAGTTTAAATAAGGCTAGTCCGTATACAACGTGAAA |
| | ACACGTGGCACCGATTCGGTGC [SEQ ID NO: 19] |
| sgRNA 2019v2-4 | GTTATAGAGCTGGAAACAGCAAAGTTATAATAAGGCTAGTCCGTATACAACGTGAAA |
| | ACACGTGGCACCGATTCGGTGC [SEQ ID NO: 20] |
| sgRNA 2019v2-5 | GTTTAAGAGCTGGAAACAGCAAAGTTTAAATAAGGCTAGTCCGTATACAACGTGGAA |
| | ACACGTGGCACCGATTCGGTGC [SEQ ID NO: 21] |
| sgRNA 2019v2-6 | GTTATAGAGCTGGAAACAGCAAAGTTATAATAAGGCTAGTCCGTATACAACGTGGAA |
| | ACACGTGGCACCGATTCGGTGC [SEQ ID NO: 22] |
| sgRNA 2019v2-7 | GTTTAAGAGCTGGAAACAGCAAAGTTTAAATAAGGCTAGTCCGTATACAACGTGGAA |
| | ACACGTGGCACCGATTCGGTGC [SEQ ID NO: 23] + G4 after CF |
| | (HR in CREATE FUSION) sequence |
| sgRNA 2019v2-8 | GTTATAGAGCTGGAAACAGCAAAGTTATAATAAGGCTAGTCCGTATACAACGTGGAA |
| | ACACGTGGCACCGATTCGGTGC [SEQ ID NO: 24] + G4 after CF |
| | (HR in CREATE FUSION) sequence |

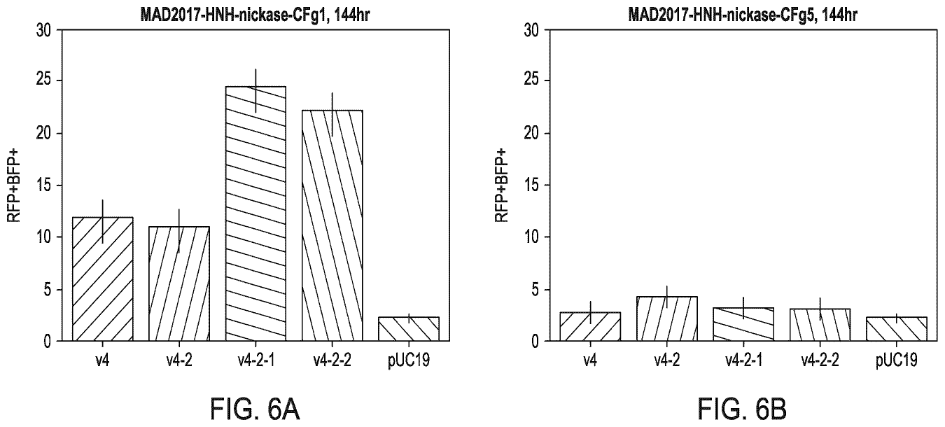

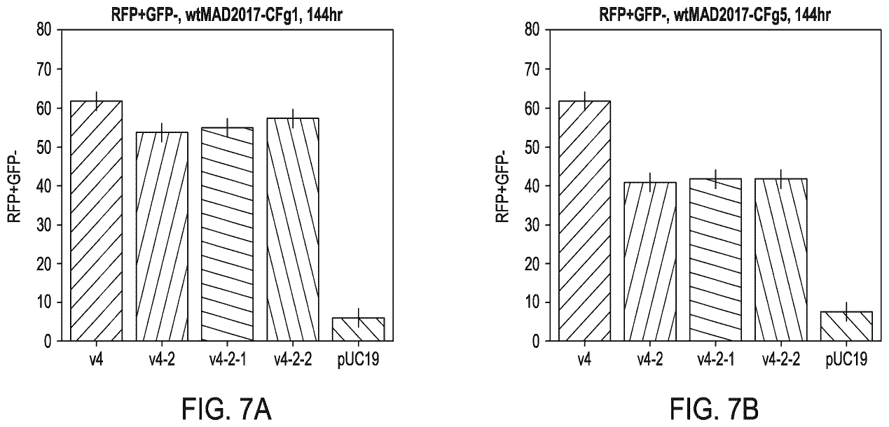

The AD2017v4 sequence was used to further optimize the gRNA scaffold. The sequences for each of these variants is shown in Table 6. The full guide comprised from 5′ to 3′: spacer+sgRNA scaffold (Table 6)+CF (HR in CREATE FUSION column in Table 4)30 G4 (if included in the scaffold design) (also in Table 4). The guides were expressed under a U6 promoter. Editing performance using the MAD2017 nickase fused to a reverse transcriptase was measured by performing in vivo editing on the GFP locus in HEK293T cells changing GFP to BFP (see the method depicted in FIG. 1B). The BFP signal was measured from the cells maintaining an RFP signal after transfection. The results are shown in FIGS. 6A and 6B. Editing performance for each gRNA scaffold was greater for the CFg1 than for CFg5 (GFPg1CF (FIG. 6A) and GFPg5CF (FIG. 6B)); however, cut activity (FIGS. 7A and 7B, GFPg1CF and GFPg5CF, respectively) with the wildtype MAD2017 nuclease indicates the presence of G4 at the 3′-terminal of CF (HR) had the greatest effect. pUC 19 is an empty cloning vector that was used for a negative control for transfection.

TABLE 6
Second Round of Optimization for MAD2017 Nickase

| Sequence Name | Sequence |
|---|---|
| sgRNA 2017v4-2 | GTTTAAGAGCTGGAAACAGCGAGTTTAAATAAGGCTTTGTCCGTACACAACTTGTAAA |
| | AGGGGCACCCGATTCGGGTGC [SEQ ID NO: 25] |
| sgRNA 2017v4-2-1 | GTTTAAGAGCTGGAAACAGCGAGTTTAAATAAGGCTTTGTCCGTACACAACTTGTAAA |
| | AGGGGCACCCGATTCGGGTGC [SEQ ID NO: 26] + G4 after CF (HR in |
| | CREATE FUSION) sequence |
| sgRNA 2017v4-2-2 | GTTTAAGAGCTGGAAACAGCGAGTTTAAATAAGGCTTTGTCCGTACACAACTTGAAAA |
| | AGGGGCACCCGATTCGGGTGC [SEQ ID NO: 27] + G4 after CF (HR in |
| | CREATE FUSION) sequence |

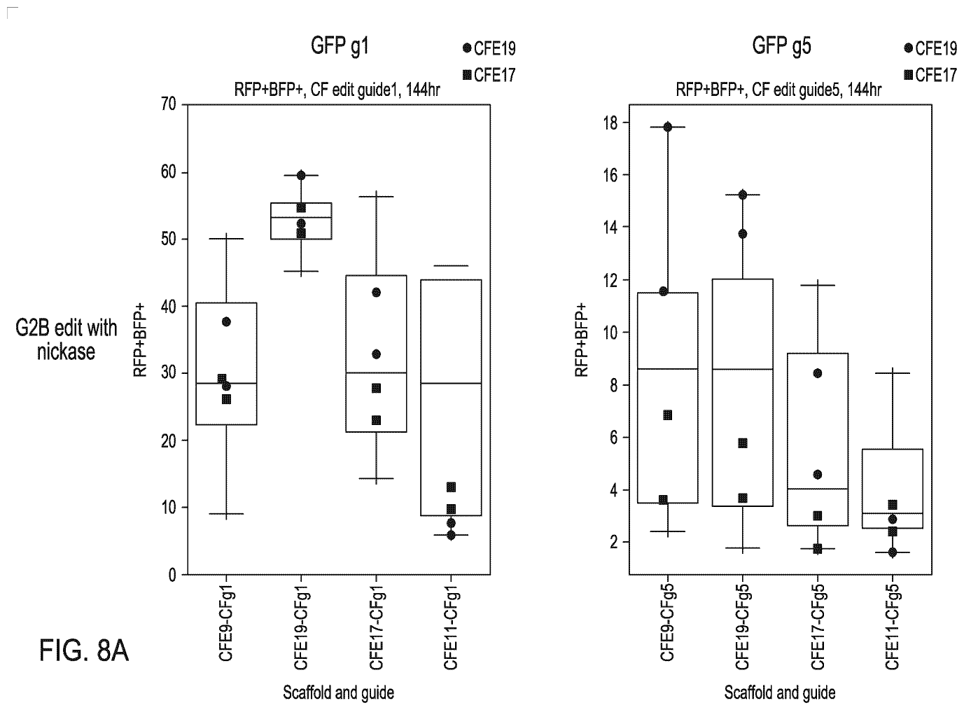

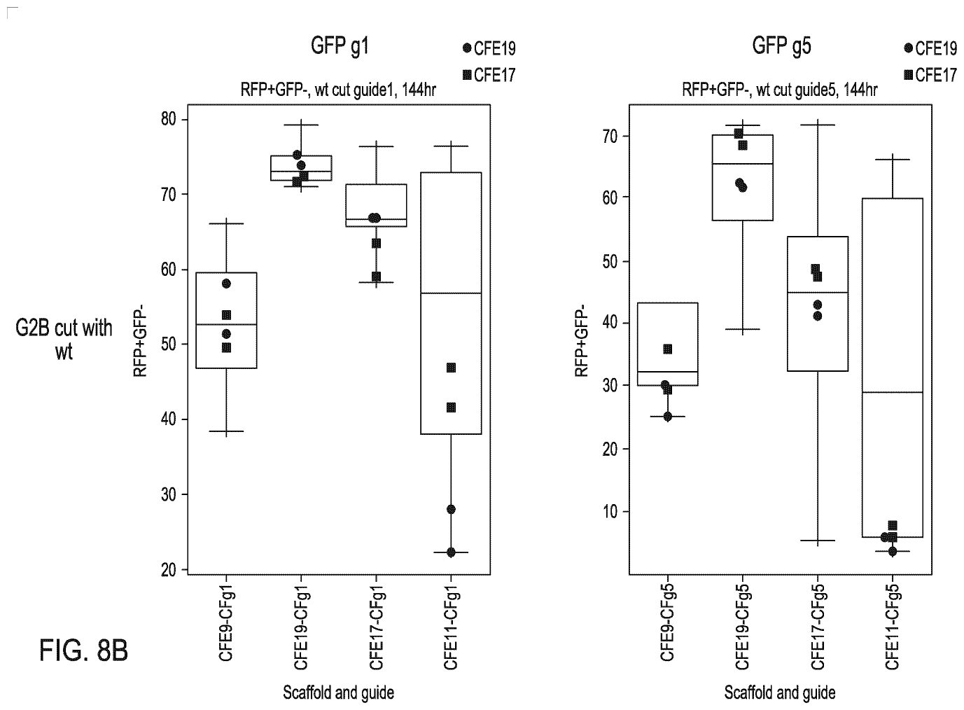

Scaffold compatibility between MAD nucleases showed that the gRNA scaffold for MAD2019 was the best universal scaffold for both MAD2019 and MAD2017 wild type and nickases. The results are shown in FIG. 8. FIG. 8A shows GFP to BFP CF editing in HEK293T cells with an integrated GFP locus (GFPg1, left top graph and GFPg5, right top graph). FIG. 8B shows GFP to GFP-cut activity in HEK293T cells with the MAD2019 and MAD2017 nucleases. CFE19-CFg1 had the best universal performance.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶ 6.

We claim:

1. A nucleic acid-guided nickase selected from the following nickases: MAD2017-H847A, having the amino acid sequence of SEQ ID NO: 5; and MAD2017-N870A. having the amino acid sequence of SEQ ID NO: 6.

2. The nickase of claim 1 having the amino acid sequence of SEQ ID NO: 5.

3. The nickase of claim 1 having the amino acid sequence of SEQ ID NO: 6.

4. The nickase of claim 1 having an amino acid sequence of SEQ ID NO: 5, in a nucleic acid-guided nickase editing system with a gRNA scaffold having a nucleic acid sequence of SEQ ID NO: 14, SEQ ID NO: 15, SEQ ID NO: 16, SEQ ID NO: 25, SEQ ID NO: 26, or SEQ ID NO: 27.

5. The nickase of claim 4 having an amino acid sequence of SEQ ID NO: 5, in the nucleic acid-guided nickase editing system with a gRNA scaffold having a nucleic acid sequence of SEQ ID NO: 14.

6. The nickase of claim 4 having an amino acid sequence of SEQ ID NO: 5, in the nucleic acid-guided nickase editing system with a gRNA scaffold having a nucleic acid sequence of SEQ ID NO: 15.

7. The nickase of claim 4 having an amino acid sequence of SEQ ID NO: 5, in the nucleic acid-guided nickase editing system with a gRNA scaffold having a nucleic acid sequence of SEQ ID NO: 16.

8. The nickase of claim 4 having an amino acid sequence of SEQ ID NO: 5, in the nucleic acid-guided nickase editing system with a gRNA scaffold having a nucleic acid sequence of SEQ ID NO: 25.

9. The nickase of claim 4 having an amino acid sequence of SEQ ID NO: 5, in the nucleic acid-guided nickase editing system with a gRNA scaffold having a nucleic acid sequence of SEQ ID NO: 26.

10. The nickase of claim 4 having an amino acid sequence of SEQ ID NO: 5, in the nucleic acid-guided nickase editing system with a gRNA scaffold having a nucleic acid sequence of SEQ ID NO: 27.

11. The nickase of claim 1 having an amino acid sequence of SEQ ID NO: 6, in a nucleic acid-guided nickase editing system with a gRNA scaffold having a nucleic acid sequence of SEQ ID NO: 14, SEQ ID NO: 15, SEQ ID NO: 16, SEQ ID NO: 25, SEQ ID NO: 26, or SEQ ID NO: 27.

12. The nickase of claim 16 having an amino acid sequence of SEQ ID NO: 6. in the nucleic acid-guided nickase editing system with a gRNA scaffold having a nucleic acid sequence of SEQ ID NO: 14.

13. The nickase of claim 16 having an amino acid sequence of SEQ ID NO: 6, in the nucleic acid-guided nickase editing system with a gRNA scaffold having a nucleic acid sequence of SEQ ID NO: 15.

14. The nickase of claim 16 having an amino acid sequence of SEQ ID NO: 6, in the nucleic acid-guided nickase editing system with a gRNA scaffold having a nucleic acid sequence of SEQ ID NO: 16.

15. The nickase of claim 16 having an amino acid sequence of SEQ ID NO: 6. in the nucleic acid-guided nickase editing system with a gRNA scaffold having a nucleic acid sequence of SEQ ID NO: 25.

16. The nickase of claim 16 having an amino acid sequence of SEQ ID NO: 4, in the nucleic acid-guided nickase editing system with a gRNA scaffold having the nucleic acid sequence of SEQ ID NO: 26.

17. The nickase of claim 16 having an amino acid sequence of SEQ ID NO: 6, in the system with a gRNA scaffold having a nucleic acid sequence of SEQ ID NO: 27.

18. The nickase of claim 1 having an amino acid sequence of SEQ ID NO: 5, in a nucleic acid-guided nickase editing system with a native CRISPR repeat having a nucleic acid sequence of SEQ ID NO: 12 and a native tracr RNA having a nucleic acid sequence of SEQ ID NO: 13.

19. The nickase of claim 1 having an amino acid sequence of SEQ ID NO: 6, in a nucleic acid-guided nickase editing system with a native CRISPR repeat having a nucleic acid sequence of SEQ ID NO: 12 and a native tracr RNA having a nucleic acid sequence of SEQ ID NO: 13.

20. The nickase of claim 1 having an amino acid sequence of SEQ ID NO: 5 or SEQ ID NO; 6, in a nucleic acid-guided nickase editing system comprising a guide RNA wherein the guide comprises from 5′ to 3′ a guide sequence, a homology region and SEQ ID NO. 30.
